(19) United States
(12) Patent Application Publication
(10) Pub. No.: US 2005/0048115 A1
(43) Pub. Date: Mar. 3, 2005

(54) Buprenorphine microspheres

(76) Inventors: Murty Mangena, Lexington, KY (US); B. Ram Murty, Lexington, KY (US)

Correspondence Address: Dr. Murty Mangena, 518 Codell Drive, Lexington, KY, 40509, US

(21) Appl. No.: 10/649,990

(22) Filed: Aug. 27, 2003

(51) Int. Cl. A61K 9/26; A61K 9/14

(52) U.S. Cl. 424/469

(57) ABSTRACT

This invention involves the establishment of manufacture of clinically useful controlled release parenteral formulation of buprenorphine hydrochloride/buprenorphine base microparticle delivery system. Buprenorphine and buprenorphine hydrochloride has been used for the treatment of pain and drug addiction. In view of minimizing the frequency of dosing and avoiding surgical procedures, controlled release parenteral dosage forms are developed using biocompatible and biodegradable polymers. These parenteral formulations also avoid oral absorption problems and potential abuse associated with other possible forms of administration such as sublingual, nasal and transdermal dosage forms. Poly-(lactic acid), poly-(glycolic acid) and their copolymers and mixture of these polymers are used for the development of microencapsulation of a buprenorphine and buprenorphine hydrochloride by solvent evaporation from oil/water emulsion. In the body, polymers are known to degrade to lactic and hydroxy-acetic acids, which are readily metabolized and eliminated.

### FIELD OF THE INVENTION

The present invention generally employs preparation of biodegradable and biocompatible polymeric microspheres/microcapsules/microparticles for controlled release of biologically active compounds. This invention is also related to the preparation of the polyester microparticles containing alkaloid like compounds, which remain chemically and physically stable and biologically active.

### BACKGROUND OF THE INVENTION

This invention deals with narcotics or opioids or alkaloids or buprenorphine or buprenorphine hydrochloride or related compounds, which can be used for pain as well as addiction treatment. Some drugs include synthetic or semi-synthetic and their derivatives in origin. This process is designed to deliver drug or combination of drugs through biocompatible polymeric matrix for defined periods of time or for controlled release with minimum to none abuse liability.

Several compounds such as buprenorphine, buprenorphine hydrochloride, methadone, normorphine, morphine, methy morphine, diprenorphine, procaine, morphine sulfate, naloxone, [D-Pen², D-Pen⁵]enkephalin, U-50, 488, methylfentanyl, butorphanol, etorphine, nalorphine, pentazocine, nalbuphine, pethidine, fentanyl, sodium bromide, cocaine, castor oil, atropine, levo-alpha-acetyl methadol (LAAM), propoxyphene, clonidine, naloxone, naltrexone, cyclazocine, pentazocine, loperamide, quartenary opiate derivatives and their related compounds are known to have medical applications. They all exhibit broad spectrum of pharmacological effects which may be used either for pain treatment or drug addiction treatment.

Thebaine is chemically the most reactive of the morphine alkaloids and contains a dienol ether system that enables it to undergo Diels-Alder reactions to produce a range of adducts in very high yields. One of the adducts of thebaine with methyl vinyl ketone is the starting point for most of the work that ultimately led the synthesis of buprenorphine. Based on structure-activity relationships in the series of tertiary alcohols derived from the thebaine-methyl vinyl ketone adducts demonstrated the importance of the structure and stereochemistry of the C₁₉ tertiary alcohol function. A wide range of candidates with different antagonist-analgesic properties have been generated by combining the knowledge of the structural relationships of different adducts and effects of piperidine N-cyclopropylmethy group. The profiles of these orvinols indicate very high affinity and intrinsic activity at both μ and κ receptors.

Opiates such as morphine produce clinically useful effects, principally analgesia and an inhibition of gastrointestinal transit, but their use is limited by side effects. The liability of many opiates to abuse and dependence is one of the side effects. A goal of opiate research has been to identify compounds that retain the useful effects of classical opiates such as morphine, but reduce or eliminate the side effects such as liability to abuse. There are three general categories of promising compounds that can be used for pain treatment or analgesics and drug addiction treatment.

Some compounds those produce clinical effects similar to morphine but act at different receptors such as κ and produce different side effects. Some κ agonists produce psychotomimetic effects that may limit their clinical utility. Second group of compounds differs from morphine primarily in their pharmacokinetics. Morphine acts at both peripheral and central receptors since it can distribute throughout the body including the central nervous system upon systemic administration. Drugs such as loperamide and quaternary opiate derivatives primarily confines to peripheral receptors and produce an inhibition of gastrointestinal transit, and possibly analgesia with less abuse liability compounds.

In the third group of compounds, buprenorphine (a semi-synthetic opioid analgesic) has come out to be the most viable compound for both pain relief and drug treatment. Buprenorphine and other drugs in this category have high affinity for μ and thus act principally at μ opioid receptors, as morphine, but they have a relatively low efficacy at these receptors. This difference in efficacy can be exploited, since different effects appear to require different levels of receptor activation. Thus, a drug could have sufficient efficacy to produce effects requiring higher levels of receptor's activation. Among opiate effects, the level of receptor activation required for analgesia depends in part on the intensity and type of the noxious stimulus present to the subject, but results with drugs such as buprenorphine suggest that relatively low levels of μ receptor activation produce clinical analgesia in humans. Buprenorphine do not produce significant respiratory depression due to low-efficacy agonist activation of μ receptor.

Buprenorphine is unusual since it is an antagonist at κ receptor so that it is characterized in vivo as a μ full or partial agonist and often classified under mixed agonist-antagonist analgesics or narcotic antagonist analgesic. Buprenorphine is an oripavine analgesic structurally related to etorphine and diprenorphine. It is set pharmacologically apart from most other opioid analgesics due to the following points. 1) It is highly lipophilic. 2) Its antinociceptive effect is readily blocked by narcotic antagonists when they are administered prior to or simultaneously with buprenorphine, but not after the antinociceptive effect is already established.

In addition to its low efficacy at μ receptors, buprenorphine shows three more important pharmacological features. The slow dissociation from the receptors contributes to the buprenorphine's long duration of action (6-10 hr). Second, buprenorphine has the same high affinity for κ receptor as for μ receptors and ten times less affinity towards δ than that of μ and κ receptors. Buprenorphine has very low efficacy at κ and δ receptors. It binds with high affinity to κ and δ opioid receptors and acts primarily as κ and δ antagonists. Finally, buprenorphine gives inverted-U shaped dose-effect curve such that intermediate doses produce bigger effects than higher doses. Thus, it exhibits autoantogonism, which limits the toxic effects of its administration in high doses. Due to buprenorphine's low efficacy at μ receptor and other pharmacological properties, it has become apparent that buprenorphine has clinical value as a maintenance drug not only for the treatment of opiate dependence, but also for the treatment of dependence on other drugs as well.

Different animal studies reveal that buprenorphine is 25-40 times more potent than morphine after parenteral administration. The physical dependence capacity of buprenorphine is of a low order. The morphine antagonist properties of buprenorphine are demonstrated in morphine-dependants. Compared to morphine, buprenorphine has a lower incidence of troublesome side effects such as pruritus and urinary retention. It displays an antitussive action against coughing, reduces heart rate, and increases spontaneous locomotor activity. The slow dissociation of buprenorphine from opioid receptors maintains homeostasis that helps to counter the development of an overt withdrawal syndrome.

Buprenorphine is used for indications for which opioids are usually prescribed. These are the “opioid-sensitive” pains, particularly acute postoperative pain, cancer pain, and certain nonmalignant pain conditions. It is a unique opioid that offers viable alternative therapy to the agonist opiates for the treatment of moderate to severe pain.

There is a small but growing body of data that support the view that powerful anxiolytic and calming agents such as buprenorphine have utility in the treatment of psychiatric disorders, depression and schizophrenia.

One of the main ideas of the drug abuse research is to replace opiates with substitutes that have no addictive properties and thus reduce or eliminate opiate abuse. In general codeine (methyl morphine) relative to morphine had little or no addiction liability, even though codein has been widely used for pain relief and cough suppression. Similarly, abuse of cocaine had waned with the introduction of the synthetic substitute procaine, which had reduced therapeutic use of cocaine as a local anesthetic.

Administration of buprenorphine to subjects not physically dependent on opioids produces morphine-like subjective and stimulus effects. No evidence has been found of dysphoric effects similar to those produced by agonist-antagonists such as nalorphine, pentazocine, and butorphanol, which are believed to act primarily through the κ system. Unlike morphine and other morphine-like agonists, buprenorphine has been administered in extremely large doses to nondependent subjects without significant depression of the cardiovascular or respiratory systems. Repeated administration of buprenorphine to the non-dependent volunteers produced a profile of effects similar to that of morphine. In general, buprenorphine substituted for and prevented the withdrawal syndrome from either morphine or methadone when the subjects who were morphine or methadone dependent, were transferred to buprenorphine. The withdrawal syndrome from the substituted buprenorphine was less when compared to morphine or methadone. The longer the period of substitution the less intense the withdrawal from the substituted buprenorphine.

Pharmacotherapies for treating dependence on narcotics have included such diverse agents as sodium bromide, cocaine, castor oil, and atropine. Compounds such as methadone, levo-alpha-acetyl methadol (LAAM), propoxyphene, clonidine, naloxone, naltrexone, and cyclazocine have been used as opiate-treatment medications. With advent of new information, Buprenorphine has been used as an alternative to methadone in pharmacotherapy for opioid addiction. Buprenorphine, similar to methadone, significantly suppresses opiod self-administration and blocks the subjective effects of full opiod agonists such as hydromorphone. Unlike methadone, buprenorphine has minimal effects on respiration. Buprenorphine also has better treatment retention rates when used as a maintenance drug in heroin addicts, and results in fewer opioid positive urine samples. Depressive symptoms were also significantly decreased in opioid addicts maintained on buprenorphine.

Initially FDA approved methadone, LAAM, and naltrexone as medications. The potential usefulness of buprenorphine as treatment medications was first studied in 1978. It was introduced as an intramuscular analgesic into medical practice in the United Kingdom in 1978 and then as a sublingual tablets in 1981. It has been proved to be safe, effective, and long lasting analgesic against moderate to severe pain in a wide variety of pain conditions. Its analgesic effectiveness has not been limited by submaximal ceiling, as was the case in several laboratory tests for antinociception.

Subsequently, the utility and effectiveness of buprenorphine as a safe analgesic and medication for opiate—as well as dual-dependants (cocaine and opiates) is established. Buprenorphine reduced self-administration by heroin-dependent men who had abused heroin for over 10 years. Buprenorphine proved to be effective for dual dependence on cocaine and opiates. However, it has been suggested that the usefulness of buprenorphine can be enhanced when it can be administered less often than once daily.

One major disadvantage to the current use of buprenorphine in detoxification program is its low and inconsistent oral absorption, making it impractical for daily oral dosing. There are sublingual tablets and transdermal dosage forms, but in countries where these have been marketed there have been cases of abuse with addicts preparing them for injection. An indictable controlled release delivery system would (1) avoid oral absorption problems, (2) circumvent the abuse problems associated with sublingual and parenteral forms and (3) addresses one of the major obstacles in agonist therapy—patient compliance.

The systemic bioavailability of buprenorphine has been estimated in several species and by various routes of administration. In female rats, the systemic bioavailability of the drug was found to be: i.v. (98%), intrarectal (54%), sublingual (13%).

Due to large hepatic and intestinal rapid “first-pass” metabolism in humans, buprenorphine displays very low systemic bioavailability following oral administration (30% by 3 h). The bioavailability, following intramuscular, sublingual, intranasal and oral administration was 40-90%, 31-58%, 48%, and 10-15% respectively. Following intravenous dosing, buprenorphine displays a distribution half-life of 2 minutes and an elimination phase half-life of 2-3 h.

Currently, Buprenorphine has been administered by oral, intramuscular, intravenous, and sublingual routes. Cylindrical long-acting 10 mg buprenorphine parenteral pellets have been prepared by compression of drug with cholesterol and glyceryl tristearate. Peak plasma concentrations of buprenorphine occurred four weeks after subcutaneous implantation in rats and plasma levels were detectable for at least twelve weeks. Implantation of such pellets requires surgical procedure. In addition, a dense fibrous compartment of such pellets that almost certainly affects drug absorption.

A new sustained release parenteral delivery system will offer advantages by increasing patient compliance as well as circumventing daily dosing, or 3 times a week dosing which is otherwise required for the opioid addiction treatment. Buprenorphine is a good candidate for a parenteral controlled release delivery system since it is potent (i.e. small dose needed), has a very short plasma half-life, is ineffective orally and requires less frequent dosing to improve patient compliance. Controlled delivery can be achieved by loading the drug into a polymer matrix, to form a microparticle.

Parenteral microparticle delivery systems may be subdivided into non-biodegradable and biodegradable systems. In addition to eventually requiring surgical removal, nondegradable implants become encapsulated by fibrous tissue, thus inhibiting further drug release. Thus, systems that ultimately disappear from the site of injection are strongly preferred. Biodegradation enables removal of the nontoxic degradation products. In addition to obviating the need to surgically remove the drug-depleted device, biodegradable systems offer simplicity of design and predictability of release.

Microspheres were developed to avoid the surgery required for the use of pellets. Microspheres are small spherical particles containing dispersed drug, which can be easily suspended in a vehicle for parenteral administration with a conventional syringe and needle. The most promising polymers for developing controlled release parenterals are the biodegradable polyesters of lactic acid (PLA), glycolic acid (PGA) and their copolymers. The homopolymers degrade more slowly than their co-polymers. Synthetic polyesters of lactic acid or lactides and glycolic acid or glycolides have been used in medical and surgical applications such as absorbable surgical implants and sutures for over several years. PLGA biodegrades by random hydrolytic cleavage (non-enzymatic) of the ester linkage into lactic acid and glycolic acid, which are metabolized by the Krebs cycle to produce carbon dioxide and water. These degradation products are expelled from the body. The in-vivo biodegradation times vary from few weeks to months depending on the molecular weight and lactide/glycolide ratio of the polymer. The 50:50 co-polymer has the shortest time for biodegradation. PLGA microspheres have also been evaluated for tissue reaction and biodistribution following intramuscular administration organs. A minima localized tissue reaction as seen on day 4 disappeared even before complete biodegradation of the PLGA matrix.

Biodegradable microsphere products can be used as parenteral controlled-release dosage forms. Microsphere products are free-flowing powders consisting of spherical particles less than 250 μm in diameter, ideally less than 125 μm. Particles of this size can be administered easily by suspending them in a suitable suspending vehicle and injecting them using a conventional syringe with an 18- or 20-gauge needle. Microspheres are also known as microcapsules, microparticles, nanoparticles, nanospheres and nanoparticles depending upon size range and location of drug distribution. Numerous sustained release drug delivery systems have been formulated using PLA, PGA and their copolymers and a wide variety of drugs have been formulated including antibiotics, polypeptides, and contraceptives.

### SUMMARY OF THE INVENTION

One of the current treatments for opiate addiction is to employ narcotic agonists and/or antagonists. Buprenorphine is a semisynthetic, highly lipophilic, potent, long acting opiate analgesic and narcotic agonist. Buprenorphine has several advantages over the other medications which include: (i) minimal physical dependence, (ii) very mild withdrawal syndrome after discontinuation, (iii) lower depressive symptoms, (iv) greater patient compliance rate when used as a maintenance drug, and (v) lower abuse liability.

Defined release profiles of buprenorphine and its salts to maintain therapeutic plasma concentration of the drug can be achieved by employing parenterally biodegradable microcapsule/microsphere delivery system. This will avoid the need for frequent drug administration and offers advantages over conventional dosage forms.

This invention covers a parenteral pharmaceutical composition designed for sustained release of a therapeutic amounts of drug over a period of time prepared in microparticle form for pain treatment as well as drug addiction treatment. The composition comprises buprenorphine base or buprenorphine hydrochloride in an effective amount and biocompatible and biodegradable polymer or mixture of polymers. Buprenorphine base or salt of buprenorphine interacts with receptor sites in mediating agonist/antagonist effects for pain and drug addiction. The supporting matrix may be a polymer comprising of homopolymers or copolymers of polylactic and galactic acids or a mixture of polymers.

The process for preparing these compositions are also disclosed, which involves solvent evaporation technique whereby the polymer is dissolved in a solvent and dispersed as oil-in-water emulsion along the drug to from microdroplets which are suspended in medium containing a dispersion agent. The process of evaporation of the solvent hardens the microparticles. The particles are then washed and dried. The dried powder can be reconstituted for injection.

### DESCRIPTION OF A PREFERRED EMBODIMENT

For the purposes of promoting an understanding of the principles of the invention, reference will now be made to the preferred embodiments thereof, and specific language will be used to describe the same. It will nevertheless be understood that no limitation of the scope of the invention is thereby intended, such alterations, modifications, and further applications of the principles of the invention being contemplated as would normally occur to one skilled in the art to which the invention relates.

With respect to the present invention, the term “phosphate buffer” refers to a buffer comprising at least one of the group consisting of PO₄³⁻, HPO₄²⁻, and H₂PO₄⁻ and at least one of the group consisting of Na⁺ and K⁺. A phosphate buffer can be made, for example, by admixing NaOH with aqueous KH₂PO₄.

With respect to the present invention, the term “halogenated organic solvent” refers to a composition consisting essentially of at least one halogenated organic solvent known in the art. Each of dichloromethane, methylene chloride, and chloroform is an example of a halogenated organic solvent.

With respect to the present invention, the term “buprenorphine” refers to at least one of the group consisting of buprenorphine free base, buprenorphine hydrochloride, and every pharmaceutically acceptable salt of buprenorphine free base.

The invention provides a method of preparing free-flowing microparticle formulations loaded with biologically active analgesics and narcotics suitable for prolonged pain and addiction treatment. In particular, the method relates to the use of polymers or combination of polymeric materials, which are biodegradable and biocompatible to obtain particles suitable for parenteral dosage forms. In an embodiment of the invention, the particles are prepared by solvent evaporation technique, which incorporates the water soluble and lipophilic forms of analgesic/narcotic compound. The following examples illustrate the processes and compositions according to this invention.

### EXAMPLE 1

### Expt.3

Preparation of Microparticles (100 mg Scale with 10% Target Drug Load, 1% CH₂Cl₂):

Microcapsules/microspheres were prepared using 50/50 poly(DL-lactide-co-glycolide), BPI and using solvent evaporation technique. Dissolve 20 mg of PVA (Avg. Mol. Wt. 30000-70000) in 2 mL of water (solution I). Dissolve 100 mg of PVA (Avg. Mol. Wt. 30000-70000) in 100 mL of water (solution II). Dissolve 91 mg of PLGA (M_{w} 60,100; Inherent Viscosity 0.7 dL/g) in 1 mL of methylene chloride using vortex mixture. To the polymer solution 9.9 mg of buprenorphine HCl was added along the solution I and vortexed/stirred for 10 seconds to obtain an oil in water emulsion. The emulsion was then added to solution II and left for stirring for three hours.

The whole slurry/suspension was centrifuged for 30 min at maximum speed using Dynac centrifuge and decanted the supernatant and washed the pallet with water three times and filtered in a cintered funnel using vacuum. The funnel was left for air drying in a vacuum desiccator. The weight of the microspheres obtained was 68.3 mg. Recovery of microspheres was 68%. Drug loading was 9.3 μg/mg of microspheres as compared to ˜100 μg/mg of microspheres.

### EXAMPLE 2

### Expt. 4, MPI # 9802-04

Preparation of Microparticles (300 mg Scale Targeted at 20% Drug Load):

Microcapsules/microspheres were prepared using 50/50 poly(DL-lactide-co-glycolide), BPI and using solvent evaporation technique as in Example 1 with certain variations. Dissolve 60 mg of PVA (Avg. Mol. Wt. 30000-70000) in 6 mL of water (solution I). Dissolve 301 mg of PVA (Avg. Mol. Wt. 30000-70000) in 300 mL of water (solution II). Dissolve 239.5 mg of PLGA (M_{w} 60,100; Inherent Viscosity 0.7 dL/g) in 3 mL of methylene chloride using vortex mixture. To the polymer solution 57.8 mg of buprenorphine HCl was added along the solution I and vortexed/stirred for 10 seconds to obtain an oil in water emulsion. The emulsion was then added drop-wise to solution II using a syringe (without needle) and left for stirring overnight.

The suspension was filtered using a cintered thimble directly without centrifugation and washed the microspheres with water several times in the thimble using vacuum. The funnel was left for air drying in a vacuum desiccator. The weight of the microspheres obtained was 243 mg. Recovery of microspheres was 81%. Mean particle size was 7.2 μm. Drug loading was 13 μg/mg of microspheres as compared to ˜200 μg/mg of microspheres.

### EXAMPLE 3

### Expt. 5, MPI # 9802-05

Preparation of Microparticles of 75/25 PLGA (300 mg Scale Targeted at 20% Drug Load):

Microcapsules/microspheres were prepared using 75/25 poly(DL-lactide-co-glycolide), BPI and using solvent evaporation technique as in Example 2 with certain variations. Dissolve 60 mg of PVA (Avg. Mol. Wt. 30000-70000) in 6 mL of water (solution I). Dissolve 303 mg of PVA (Avg. Mol. Wt. 30000-70000) in 300 mL of water (solution II). Dissolve 239 mg of PLGA (M_{w} 97,000; Inherent Viscosity 0.67 dL/g) in 3 mL of methylene chloride using vortex mixture. To the polymer solution 58 mg of buprenorphine HCl was added along the solution I and vortexed/stirred for 15 seconds to obtain an oil in water emulsion. The emulsion was then added dropwise to solution II using a syringe and 23 gauge needle and left for stirring overnight.

The suspension was centrifuged at 3600 RPM for 30 min. and washed three times and filtered using a cintered funnel using vacuum. The funnel was left for air drying in a vacuum desiccator. The weight of the microspheres obtained was 219 mg. Recovery of microspheres was 74%. Mean particle size was 14 μm. Drug loading was 21 μg/mg of microspheres as compared to ˜200 μg/mg of microspheres.

### EXAMPLE 4

### Expt. 6, MPI #9802-06

Preparation of Microparticles (300 mg Scale Targeted at 10% Drug Load, 2% CH₂Cl₂):

Microcapsules/microspheres were prepared using 50/50 poly(DL-lactide-co-glycolide), BPI and using solvent evaporation technique as in Example 3 with certain variations. Dissolve 58.9 mg of PVA (Avg. Mol. Wt. 30000-70000) in 6 mL of water (solution I). Dissolve 299.8 mg of PVA (Avg. Mol. Wt. 30000-70000) in 300 mL of water (solution II). Dissolve 270.7 mg of PLGA (M_{w} 60,100; Inherent Viscosity 0.7 dL/g) in 6 mL of methylene chloride using vortex mixture. To the polymer solution 29.7 mg of buprenorphine HCl was added along the solution I and vortexed/stirred for 15 seconds to obtain an oil in water emulsion. The emulsion was added dropwise to solution II using a syringe and needle and left for stirring overnight.

The suspension was centrifuged at 3600 RPM for 30 min and washed three times and filtered using a cintered funnel using vacuum. The funnel was left for air drying in a vacuum desiccator. The weight of the microspheres obtained was 265 mg. Recovery of microspheres was 88%. Mean particle size was 8.6 μm. Drug loading was 2.1 μg/mg of microspheres as compared to ˜100 μg/mg of microspheres.

### EXAMPLE 5

### Expt. 9, MPI #9802-09

Preparation of Microparticles (300 mg Scale Targeted at 30% Drug Load, 2% CH₂Cl₂):

Microcapsules/microspheres were prepared using 50/50 poly(DL-lactide-co-glycolide), BPI and using solvent evaporation technique as in Example 4 with certain variations. Dissolve 61.4 mg of PVA (Avg. Mol. Wt. 30000-70000) in 6 mL of water (solution I). Dissolve 301 mg of PVA (Avg. Mol. Wt. 30000-70000) in 300 mL of water (solution II). Dissolve 210.4 mg of PLGA (M_{w} 60,100; Inherent Viscosity 0.7 dL/g) in 6 mL of methylene chloride using vortex mixture. To the polymer solution 91.3 mg of buprenorphine HCl was added along the solution I and vortexed/stirred for 15 seconds to obtain an oil in water emulsion. The emulsion was then added dropwise to solution II using a syringe and needle and left for stirring overnight.

The suspension was centrifuged at 3600 RPM for 30 min and filtered and washed several times with water. Before centrifugation, the microsphere suspension was subjected to rotary evaporation for 2 h at 37° C. The funnel was left for air drying in a vacuum desiccator. The weight of the microspheres obtained was 203 mg. Recovery of microspheres was 67%. Mean particle size was 4.1 μm. Drug loading was 6 μg/mg of microspheres as compared to ˜300 μg/mg of microspheres.

### EXAMPLE 6

### Expt. 10, MPI #9802-10

Preparation of Microparticles with 75/25 PLGA (300 mg Scale Targeted at 10% Drug Load, 2% CH₂Cl₂):

Microcapsules/microspheres were prepared using 75/25 poly(DL-lactide-co-glycolide), BPI and using solvent evaporation technique as in Example 5 with certain variations. Dissolve 60.3 mg of PVA (Avg. Mol. Wt. 30000-70000) in 6 mL of water (solution I). Dissolve 300.3 mg of PVA (Avg. Mol. Wt. 30000-70000) in 300 mL of water (solution II). Dissolve 268.3 mg of PLGA (M_{w} 97,400; Inherent Viscosity 0.67 dL/g) in 6 mL of methylene chloride using vortex mixture. To the polymer solution 30.4 mg of buprenorphine HCl was added along the solution I and vortexed/stirred for 15 to obtain an oil in water emulsion. The emulsion was then added dropwise to solution II using a syringe and needle and left for stirring overnight.

The suspension was centrifuged at 3600 RPM for 30 min and filtered and washed several times with water. Before centrifugation, the microsphere suspension was subjected to rotary evaporation for 2 h at 37° C. The funnel was left for air drying in a vacuum desiccator. The weight of the microspheres obtained was 261 mg. Recovery of microspheres was 88%. Mean particle size was 8.3 μm. Drug loading was 4.9 μg/mg of microspheres as compared to ˜100 μg/mg of microspheres.

### EXAMPLE 7

### Expt. 11, MPI #9802-11

Preparation of Microparticles (300 mg Scale Targeted at 10% Drug Load, 0.5% CH₂Cl₂):

Microcapsules/microspheres were prepared using 50/50 poly(DL-lactide-co-glycolide), BPI and using solvent evaporation technique as in Example 6 with certain variations. Dissolve 61 mg of PVA (Avg. Mol. Wt. 30000-70000) in 6 mL of water (solution 1). Dissolve 301 mg of PVA (Avg. Mol. Wt. 30000-70000) in 300 mL of water (solution II) present in 1000 mL beaker containing a stir bar (10×38 mm). Dissolve 270.6 mg of PLGA (M_{w} 60,100; Inherent Viscosity 0.7 dL/g) in 1.5 mL of methylene chloride using vortex mixture. To the polymer solution 29.2 mg of buprenorphine HCl was added along the solution I and vortexed/stirred for 15 to obtain an oil in water emulsion. The emulsion was then added dropwise to solution II using a syringe and needle and left for stirring overnight.

The suspension was centrifuged at 3600 RPM for 15 min and washed twice with water. After that it was again centrifuged final wash with water was done during filtration. The filtration was done using Millipore membrane (0.65 μm). The membrane containing the microspheres was left for air drying in a vacuum desiccator. The weight of the microspheres obtained was 266 mg. Recovery of microspheres was 89%. Mean particle size was 7.2 μm. Drug loading was 26.2 μg/mg of microspheres as compared to ˜100 μg/mg of microspheres.

### EXAMPLE 8

### Expt. 12, MPI #9802-12

Preparation of Microparticles (300 mg Scale Targeted at 10% Drug Load, 0.5% CH₂Cl₂) by dissolving the active in the aqueous solution:

Microcapsules/microspheres were prepared using 50/50 poly(DL-lactide-co-glycolide), BPI and using solvent evaporation technique as in Example 7 with certain variations. Dissolve 60 mg of PVA (Avg. Mol. Wt. 30000-70000) in 6 mL of water (solution I). Dissolve 300 mg of PVA (Avg. Mol. Wt. 30000-70000) in 300 mL of water (solution II). Dissolve 272 mg of PLGA (M_{w} 60,100; Inherent Viscosity 0.7 dL/g) in 1.5 mL of methylene chloride using vortex mixture. To the solution I, 30.2 mg of buprenorphine HCl was added. This solution was again added to the polymer solution as in Example 7. The emulsion was added dropwise to solution II using a syringe and needle and left for stirring overnight.

The suspension was centrifuged at 3600 RPM for 15 min and washed twice with water. After that it was again centrifuged final wash with water was done during filtration. The filtration was done using Millipore membrane (0.65 μm). The membrane containing the microspheres was left for air drying in a vacuum desiccator. The weight of the microspheres obtained was 260 mg. Recovery of microspheres was 86%. Mean particle size was 8.2 μm. Drug loading was 23.2 μg/mg of microspheres as compared to ˜100 μg/mg of microspheres.

### EXAMPLE 9

### Expt. 13, MPI #9803-13

Preparation of Microparticles to Verify the Reproducibility of Example 7:

Microcapsules/microspheres were prepared using 50/50 poly(DL-lactide-co-glycolide), BPI and using solvent evaporation technique as in Example 7 to verify the reproducibility. Dissolve 63.5 mg of PVA (Avg. Mol. Wt. 30000-70000) in 6 mL of water (solution I). Dissolve 300.6 mg of PVA (Avg. Mol. Wt. 30000-70000) in 300 mL of water (solution II). Dissolve 273.9 mg of PLGA (M_{w} 60,100; Inherent Viscosity 0.7 dL/g) in 1.5 mL of methylene chloride using vortex mixture. To the polymer solution 30.2 mg of buprenorphine HCl was added along the solution I and vortexed/stirred for 15 seconds to obtain an oil in water emulsion. The emulsion was then added dropwise to solution II using a syringe and needle and left for stirring overnight.

The suspension was centrifuged at 3600 RPM for 15 min and the supernatant filtered using Millipore membrane (0.65 μm). The pellet was suspended in water and it was centrifuged twice and filtered through the membrane. The membrane containing the microspheres was left for air drying in a vacuum desiccator. The weight of the microspheres obtained was 271 mg. Recovery of microspheres was 89%. Mean particle size was 3.4 μm. Drug loading was 22.2 μg/mg of microspheres as compared to ˜100 μg/mg of microspheres.

The examples from 1 through 9 contained the same source of Buprenorphine HCl (MPI # C-96-012).

### EXAMPLE 10

### Expt. 14, MPI #9803-14

Preparation of Microparticles (300 mg Scale Targeted at 10% Drug Load, 0.5% CH₂Cl₂) using a Different Source of Active:

Microcapsules/microspheres were prepared using 50/50 poly(DL-lactide-co-glycolide), BPI and using solvent evaporation technique as in Example 9, but employing a different source of buprenorphine HCl (C-98001). Dissolve 60.7 mg of PVA (Avg. Mol. Wt. 30000-70000) in 6 mL of water (solution I). Dissolve 300.5 mg of PVA (Avg. Mol. Wt. 30000-70000) in 300 mL of water (solution II). Dissolve 269.5 mg of PLGA (M_{w} 60,100; Inherent Viscosity 0.7 dL/g) in 1.5 mL of methylene chloride using vortex mixture. To the polymer solution 30.7 mg of buprenorphine HCl was added along the solution I and vortexed/stirred for 15 seconds to obtain an oil in water emulsion. The emulsion was then added dropwise to solution II using a syringe and needle and left for stirring overnight.

The suspension was centrifuged at 3600 RPM for 15 min and the supernatant was filtered using Millipore membrane (0.65 μm). The pellet was suspended in water and it was centrifuged twice and filtered through the membrane. The membrane containing the microspheres was left for air drying in a vacuum desiccator. The weight of the microspheres obtained was 271 mg. Recovery of microspheres was 90%. Mean particle size was 4.3 μm. Drug loading was 25.1 μg/mg of microspheres as compared to ˜100 μg/mg of microspheres.

### EXAMPLE 11

### Expt. 16, MPI #9803-16

Preparation of Microparticles (900 mg Scale Targeted at 10% Drug Load, 0.5% CH₂Cl₂):

Microcapsules/microspheres were prepared using 50/50 poly(DL-lactide-co-glycolide), BPI and using solvent evaporation technique. Dissolve 180 mg of PVA (Avg. Mol. Wt. 30000-70000) in 18 mL of water (solution I) in a 25 mL beaker. Dissolve 901 mg of PVA (Avg. Mol. Wt. 30000-70000) in 900 mL of water (solution II) present in 2000 mL containing a stir bar (10×52 mm). Dissolve 810 mg of PLGA (M_{w} 60,100; Inherent Viscosity 0.7 dL/g) in 4.5 mL of methylene chloride and stirred/vortexed to dissolve in a 20-mL beaker. To the polymer solution 93.6 mg of buprenorphine HCl was added along the solution I. It was homogenized using a flat bottom 7 mm generator attached to Powergen 700 at setting 4 (˜10,000 RPM) for 1 min to obtain an oil in water emulsion. About 10 mL of the PVA solution (II) was set aside. The emulsion was then added dropwise using a syringe and needle to the bulk of the PVA solution (II) with continuous stirring at maximum stirrer speed. The beaker with the residual emulsion is rinsed with the 10 mL of solution II and transferred to the dilute microsphere suspension. The suspension was left for stirring overnight.

The suspension was centrifuged at 3600 RPM for 15 min and the supernatant filtered using Millipore membrane (0.65 μm). The pellet was suspended in water and it was centrifuged twice and filtered through the membrane. The membrane containing the microspheres was left for air drying in a vacuum desiccator. The weight of the microspheres obtained was 903.6 mg. Recovery of microspheres was 87.5%. Mean particle size was 4.3 μm. Drug loading was 26 μg/mg of microspheres as compared to ˜100 μg/mg of microspheres.

### EXAMPLE 12

### Expt. 20, MPI #9805-20

Preparation of Microparticles with Low M_{w} PLGA (900 mg Scale Targeted at 10% Drug Load, 0.3% CH₂Cl₂):

Incorporation of buprenorphine HCl in low molecular weight 50/50 poly(DL-lactide-co-glycolide), BPI and using solvent evaporation technique as in Example 11 with certain modifications. Dissolve 180 mg of PVA (Avg. Mol. Wt. 30000-70000) in 18 mL of water (solution I). Dissolve 900 mg of PVA in 900 mL of water (solution II) present in 2000 mL containing a stir bar (10×52 mm). Dissolve 810 mg of PLGA (M_{w} 6,630; Inherent Viscosity 0.16 dL/g) in 3.0 mL of methylene chloride using sonicator and hand swirling for 30 min. To the polymer solution 90 mg of buprenorphine HCl was added along the solution I. Rest of the procedure is the same as given in Example 11.

The weight of the recovered PLGA/buprenorphine HCl was 690 mg. Recovery was 76%. Incorporation of buprenorphine HCl was 17.9 μg/mg of the preparation.

### EXAMPLE 13

### Expt. 21, MPI #9805-21

Preparation of Microparticles using Mixture of PLGAs (900 mg Scale Targeted at 10% Drug Load, 0.3% CH₂Cl₂):

Microcapsules/microspheres were prepared using combination of 50/50 poly(DL-lactide-co-glycolide) of BPI and using solvent evaporation technique as in Example 11 with certain modifications. Dissolve 181 mg of PVA (Avg. Mol. Wt. 30000-70000) in 18 mL of water (solution I). Dissolve 901 mg of PVA (Avg. Mol. Wt. 30000-70000) in 900 mL of water (solution II) present in 2000 mL containing a stir bar (10×52 mm). Dissolve 404 mg of PLGA (M_{w} 6,630; Inherent Viscosity 0.16 dL/g) plus 400 mg of another PLGA (M_{w} 54,100; Inherent Viscosity 0.64 dL/g) in 3.0 mL of methylene chloride using sonicator and hand swirling for 30 min. To the polymer solution 92.3 mg of buprenorphine HCl was added along the solution I. Rest of the procedure is the same as given Example 11.

The weight of the microspheres obtained was 675 mg. Recovery of microspheres was 75%. Mean particle size was 6.6 μm. Drug loading was 24.9 μg/mg of microspheres as compared to ˜100 μg/mg of microspheres.

### EXAMPLE 14

### Expt. 22, MPI #9805-22

Preparation of Microparticles (300 mg Scale Targeted at 10% Drug Load, 0.7% Ethyl Acetate):

Microcapsules/microspheres were prepared using 50/50 poly(DL-lactide-co-glycolide) of BPI and using solvent evaporation technique as in Example 11 with certain modifications. Dissolve 61.9 mg of PVA (Avg. Mol. Wt. 30000-70000) in 6 mL of water (solution I) in a10 mL beaker. Dissolve 299 mg of PVA (Avg. Mol. Wt. 30000-70000) in 300 mL of water (solution II) present in 700 mL. Dissolve 268.6 mg of PLGA (Mw 54,100; Inherent Viscosity 0.64 dL/g) in 2.0 mL of ethyl acetate using vortex. The polymer solution was made in a 20-mL screw cap tube. To the polymer solution 30 mg of buprenorphine HCl was added. Solution I was then added to the polymer suspension and vortexed for 1 min. to form an emulsion. About 10 mL of the PVA solution (II) was set aside. The emulsion was added dropwise using a syringe and needle to the bulk of the PVA solution (II) with continuous stirring at maximum stirrer speed. The beaker with the residual emulsion is rinsed with the 10 mL of solution II and transferred to the dilute microsphere suspension. Stirring was carried out overnight. The microspheres were allowed to settle for an hour before centrifugation. The whole medium was centrifuged at 3600 RPM for 15 min. using Mistral centrifuge. The supernatant was filtered through a preweighed 0.65 μm membrane filter. The microspheres were air-dried under vacuum filtration and finally dried in a vacuum dessicator for overnight.

The weight of the microspheres obtained was 298 mg. Recovery of microspheres was 87.6%. Mean particle size was 6.8 μm. Drug loading was 17.6 μg/mg of microspheres as compared to ˜100 μg/mg of microspheres.

### EXAMPLE 15

### Expt. 23, MPI #980601R

Preparation of Microparticles using Mixture of PLGAs and Dried by Lyophilization (900 mg Scale Targeted at 10% Drug Load, 0.3% CH₂Cl₂):

Microcapsules/microspheres were prepared using combination of 50/50 poly(DL-lactide-co-glycolide) of BPI and using solvent evaporation technique as in Example 13 with certain modifications. Polyvinyl alcohol (PVA) 180.6 mg was weighed and transferred into a 25 mL beaker containing 18 mL of nanopure water (Solution I) taken in a 25 mL beaker. The suspension was stirred until dissolved. In another 2 L beaker was weighed 901.8 mg of PVA and added 900 ml of nanopure water (Solution II). The suspension was stirred until dissolved. PLGA polymers; 397.4 mg (M_{w} 54,100; Inherent Viscosity 0.64 dL/g) plus 401.2 mg of another PLGA (M_{w} 6,630; Inherent Viscosity 0.16 dL/g) were mixed and transferred into a 20 mL beaker. To the beaker was added 3 mL of CH₂Cl₂. The mixture was stirred and vortexed until polymers were dissolved. Buprenorphine hydrochloride was weighed accurately (88.3 mg) and added to the beaker containing PLGA polymer solution.

Solution I was then added to the polymer suspension and homogenized as in Example 11 for 1 min. to form an emulsion. About 10 mL of the PVA solution (II) was set aside. The emulsion was added dropwise using a syringe and needle to the bulk of the PVA solution (II) with continuous stirring at maximum stirrer speed. The beaker with the residual emulsion is rinsed with the 10 mL of solution II and transferred to the dilute microsphere suspension. Stirring was carried out through overnight. The microspheres were allowed to settle for an hour before centrifugation. The whole medium was centrifuged at 3600 RPM for 15 min. using Mistral centrifuge. The supernatant was decanted and the pellet was resuspended in water and centrifuged. The washing procedure was repeated twice. The final pellet/suspension was placed on a petriplate and subjected to lyophilization using VirTis Unitop 200.

The weight of the microspheres recovered was 798 mg. The yield of lyophilized microspheres was 78%. The distribution of microsphere particle size was determined using Scanning electron microscopy. The particle size range for the microspheres was found to be ˜2-50μ. Drug loading was 22.8 μg/mg of microspheres as compared to ˜100 μg/mg of microspheres.

### EXAMPLE 16

### Expt. 26, MPI #980803R

Preparation of Microparticles using Mixture of PLGAs (3.6 g Scale Targeted at 10% Drug Load, 0.3% CH₂Cl₂):

Microcapsules/microspheres were prepared using combination of 50/50 poly(DL-lactide-co-glycolide) of BPI and using solvent evaporation technique as in Example 15 with certain modifications. The 720.0 mg of polyvinyl alcohol (PVA) was weighed and transferred into a 100 mL beaker containing 72 mL of nanopure water (Solution I). The suspension was stirred until dissolved. The 3.6 g of PVA was weighed in another 4 L beaker, and 3600 mL of nanopure water was added (Solution II). The suspension was stirred until dissolved. PLGA polymers, 1.620 g (Mw 6,630 and viscosity 0.16 dL/g) and 1.623 g (Mw 54,100 and viscosity 0.64 dL/g) were mixed and transferred into a 100 mL beaker and 12 mL of CH₂Cl₂ was added to the beaker. The mixture was stirred and sonicated until polymers were dissolved. Buprenorphine hydrochloride was weighed accurately (360.0 mg) and added to the beaker containing the PLGA polymer solution. The PVA solution I was then added to the polymer suspension. The suspension was homogenized using a Powergen 700 homogenizer (speed set at 4) for 1 min. to form an emulsion. About 80 mL of the PVA solution II was set aside. The emulsified suspension was then added dropwise using a syringe to the bulk of the PVA solution II, dispersed using a 35 mm power generator (speed set at 3) for 20 min., and then stirred using a stirrer bar at maximum speed. Processing of the microspheres was carried out as given in example 15.

The microspheres were then left in a freezer at −20° C. for overnight and lyophilized as in Example 15. Total amount of microspheres recovered was 3.1 g. The yield of lyophilized microspheres was 87%. The distribution of microsphere particle size was determined using a Hyac Royco particle counter. The maximum particle size population was found to be between 2-10μ, and the particle size was determined to be less than ˜50μ. Drug loading was 21.3 μg/mg of microspheres as compared to ˜100 μg/mg of microspheres.

### EXAMPLE 17

### EXPT. 27, MPI #980901R

Preparation of Microparticles using Mixture of PLGAs and pH Adjustment of the Medium (900 mg Scale Targeted at 10% Drug Load, 0.3% CH₂Cl₂):

Microcapsules/microspheres were prepared using combination of 50/50 poly(DL-lactide-co-glycolide) of BPI and using solvent evaporation technique as in Example 15 with certain modifications. Polyvinyl alcohol (PVA) 180 mg was weighed and transferred into a 20 mL beaker containing 18 mL of nanopure water (Solution I) and 0.5 N NaOH was added to bring the pH to 9.0. The suspension was stirred until dissolved. In another 2 L beaker was weighed 900 mg of PVA and added 900 ml of nanopure water (Solution II) and 0.5 N NaOH was added to bring the pH to 9.0. The suspension was stirred until dissolved. PLGA polymers; 404.5 mg (M_{w} 54,100; Inherent Viscosity 0.64 dL/g) plus 405 mg of another PLGA (M_{w} 6,630; Inherent Viscosity 0.16 dL/g) were mixed and transferred into a 25 mL beaker. To the beaker was added 3 mL of CH₂Cl₂. The mixture was stirred and vortexed until polymers were dissolved. Buprenorphine hydrochloride was weighed accurately (89.1 mg) and added to the beaker containing PLGA polymer solution. The beaker with the residual emulsion is rinsed with the 20 mL solution II (which was set aside before) and transferred to the dilute microsphere suspension. Processing was done as given Example 15. The suspension was left at −20° C. for three days and then lyophilized as in Example 15.

Total amount of microspheres recovered was 685 mg. The yield of lyophilized microspheres was 76%. Mean particle size was 8.7 μm Drug loading was 26.9 μg/mg of microspheres as compared to ˜100 μg/mg of microspheres.

### EXAMPLE 18

### EXPT. 28, MPI #981001R

Preparation of Microparticles using Mixture of PLGAs and Phosphate Buffer pH 7.4 (900 mg Scale Targeted at 10% Drug Load, 0.3% CH₂Cl₂):

Microcapsules/microspheres were prepared using combination of 50/50 poly (DL-lactide-co-glycolide) of BPI and using solvent evaporation technique as in Example 15 with certain modifications. Polyvinyl alcohol (PVA) 180.4 mg was weighed and transferred into a 25 mL beaker containing 18 mL of potassium phosphate buffer pH 7.4 (Solution I). The suspension was stirred until dissolved. In another 2 L beaker was weighed 899.6 mg of PVA and added 900 ml of potassium phosphate buffer pH 7 (Solution II). The suspension was stirred until dissolved. PLGA polymers; 405 mg (M_{w} 54,100; Inherent Viscosity 0.64 dL/g) plus 405 mg of another PLGA (M_{w} 6,630; Inherent Viscosity 0.16 dL/g) were mixed and transferred into a 25 mL beaker. To the beaker was added 3 mL of CH₂Cl₂. The mixture was stirred and vortexed until polymers were dissolved. Buprenorphine hydrochloride was weighed accurately (89.5 mg) and added to the beaker containing PLGA polymer solution. The beaker with the residual emulsion is rinsed with the 20 mL solution II (which was set aside before) and transferred to the dilute microsphere suspension. Processing was done as given Example 15.

Total amount of microspheres recovered was 760 mg. The yield of lyophilized microspheres was 84%. Mean particle size was 4.8 μm. Drug loading was 36.3 μg/mg of microspheres as compared to ˜100 μg/mg of microspheres.

### EXAMPLE 19

Conversion of Buprenorphine Hydrochloride (0.4 g scale) to Buprenorphine Base:

Since, the buprenorphine free base is more lipophilic than the buprenorphine HCl, the acid form was converted to its base form to enhance drug loading into the polymer.

Preparation of Buprenorphine Free Base: Placed 416 mg of buprenorphine hydrochloride (MPI # C98001) in a beaker. Added 50 mL of nanopure water to the beaker with continuous stirring with a magnetic stirring bar. Stirring is continued until clear solution was obtained. Adjusted pH of the solution between 7.0 and 7.5 by the addition of 2N sodium hydroxide solution until white precipitate was formed. Added 10 mL of methylene chloride to the above suspension. Stirred the suspension until all of the precipitate dissolved. Transferred the solution into a 125 mL of separatory funnel. Separated the organic layer into a beaker. Extracted the aqueous layer in the separatory funnel with 2×15 mL of methylene chloride. Pooled the entire organic layer into a flask. Added calcium chloride to the combined extract and filtered the above mixture through a filter paper. The filtrate was evaporated to dryness using rotary evaporator and the white solid was then vacuum dried. Yield of the base was 80%. Characterized the solid by melting point (209° C.) which corresponded to the melting point of buprenorphine free base.

### EXAMPLE 20

### Expt. 29, MPI #981101R

Preparation of Microparticles using Mixture of PLGAs, Buprenorphine Base and Phosphate Buffer pH 7.4 (900 mg Scale Targeted at 10% Drug Load, 0.3% CH₂Cl₂):

Microcapsules/microspheres were prepared using combination of 50/50 poly (DL-lactide-co-glycolide) of BPI and using solvent evaporation technique as in Example 15 with certain modifications. The 180.0 mg of polyvinyl alcohol (PVA) was weighed and transferred into a 25 mL beaker containing 18 mL of phosphate buffer, pH 7.4 (Solution I). The suspension was stirred until dissolved. The 900.0 mg of PVA was weighed in another 2 L beaker, and 900 mL of phosphate buffer (pH 7.4) was added (Solution II). The suspension was stirred until dissolved. PLGA polymers, 404.0 mg (Viscosity, 0.16 dL/g and Mw 6,630) and 405.0 mg (Viscosity, 0.64 dL/g and Mw 54,100) were mixed and transferred into a 25 mL beaker. Added 3 mL of CH₂Cl₂ to the beaker. The mixture was stirred and sonicated until polymers were dissolved. Buprenorphine free base, which was prepared in Example 19, was weighed accurately (89.3 mg) and added to the beaker containing the PLGA polymer solution. Rest of the procedure was the same as in Example 15.

The suspension was stirred for ˜3 h. The microspheres were allowed to settle for 1 h. The microspheres were then centrifuged for 15 min. at 3600 rpm using a Mistral centrifuge. The supernatant liquid was decanted, and the microspheres were washed 3 times with nanopure water. The microspheres were transferred into petriplates and lyophilized as in Example 15. The yield of lyophilized microspheres was 808 mg. The yield was 89%. The percentage incorporation of buprenorphine in the microspheres was analyzed by HPLC and was found to be ˜81.8 μg of buprenorphine/mg of microspheres as compared to ˜100 μg/mg of microspheres.

### EXAMPLE 21

### Expt. 30, MPI #981201R

Reproduction of Example 20 with 9% Target Drug Load:

Microcapsules/microspheres were prepared using combination of 50/50 poly (DL-lactide-co-glycolide) of BPI and using solvent evaporation technique as in Example 20 to verify the reproducibility of the process. The 180.0 mg of polyvinyl alcohol (PVA) was weighed and transferred into a 25 mL beaker containing 18 mL of phosphate buffer, pH 7.4 (Solution I). The suspension was stirred until dissolved. Another 900.0 mg of PVA was weighed in a 2 L beaker, and 900 mL of phosphate buffer (pH 7.4) was added. The suspension was stirred until dissolved. PLGA polymers, 410.6 mg (Viscosity, 0.16 dL/g and Mw 6,630) and 414.3 mg (Viscosity, 0.64 dL/g and Mw 54,100), were mixed and transferred into a 25 mL beaker. Added 3 mL of CH₂Cl₂ to the beaker. The mixture was stirred and sonicated until polymers were dissolved. Buprenorphine free base, which was prepared in Example 19 was weighed accurately (80.0 mg) and added to the beaker containing the PLGA polymer solution. Solution mixing was the same as in example 15. The suspension was stirred for 3 h. The microspheres were allowed to settle for overnight. The microspheres were then centrifuged for 15 min. at 3600 rpm using a Mistral centrifuge. The supernatant liquid was decanted, and the microspheres were washed 3 times with nanopure water. The microspheres were transferred into petriplates, frozen for ˜3 h at −40° C. and then lyophilized as in example 15.

The yield of lyophilized microspheres was 793 mg. The microspheres yield was 88%. The percentage incorporation of buprenorphine in the microspheres was analyzed by HPLC and was found to be ˜72 μg of buprenorphine/mg of Microspheres, as compared to ˜90 μg/mg.

### EXAMPLE 22

Conversion of Buprenorphine Hydrochloride (2.2 g Scale) to Buprenorphine Base:

Preparation of buprenorphine base in large quantity and also to verify the reproducibility of the Example 19. Placed 2.225 g of buprenorphine hydrochloride (MPI # C98001) in a beaker. Added 175 mL of water for injection to the beaker with continuous stirring using a magnetic stirring bar. Stirring is continued until clear solution was obtained. Adjusted pH of the solution between 7.0 and 7.5 by the addition of 2N sodium hydroxide solution until white precipitate was formed. Added 40 mL of methylene chloride to the above suspension. Stirred the suspension until all of the precipitate dissolved. Transferred the solution in into a 250 mL of separatory funnel. Separated the organic layer into a beaker. Extracted the aqueous layer in the separatory funnel with 2×10 mL of methylene chloride. Pooled the entire extracted organic layer into one flask. Added calcium chloride to the combined extract and filtered the above mixture through a filter paper. The filtrate was evaporated to dryness using rotary evaporator (3-4 h) and the white solid was then vacuum dried (˜24 h). The yield of the base 96% and melting point was 209° C.

### EXAMPLE 23

### Expt. 31, MPI #990201R

Reproduction of Example 20 with 10% Target Drug Load with Different Lots of PLGAs and Active:

Microcapsules/microspheres were prepared using combination of 50/50 poly (DL-lactide-co-glycolide) of BPI and using solvent evaporation technique as in Example 21 to verify the process variability with different lots of key materials. The 180.0 mg of polyvinyl alcohol (PVA) was weighed and transferred into a 25 mL beaker containing 18 mL of phosphate buffer, pH 7.4 (Solution I). The suspension was stirred until dissolved. Another 900.0 mg of PVA was weighed in a 2 L beaker, and 900 mL of phosphate buffer (pH 7.4) was added. The suspension was stirred until dissolved. PLGA polymers, 403 mg (Viscosity, 0.66 dL/g and Mw 53,600) and 402 mg (Viscosity, 0.2 dL/g and Mw 9,440), were mixed and transferred into a 25 mL beaker. Added 3 mL of CH₂Cl₂ to the beaker. The mixture was stirred and sonicated until polymers were dissolved. Buprenorphine free base, which was prepared in Example 22 was weighed accurately (94.4 mg) and added to the beaker containing the PLGA polymer solution. Solution mixing was the same as in example 21. The suspension was kept at 25° C. and stirred at 900 RPM overnight. The microspheres were allowed to settle for one hour. The microspheres were then centrifuged for 15 min. at 3600 rpm using a Mistral centrifuge. The supernatant liquid was decanted, and the microspheres were washed 3 times with nanopure water. The microspheres were transferred into petriplates, frozen for ˜2 h in the lyophilization chamber and then lyophilized.

The yield of lyophilized microspheres was 761 mg. The microspheres yield was 88%. The percentage incorporation of buprenorphine in the microspheres was analyzed by HPLC and was found to be 84.7 μg of buprenorphine/mg of Microspheres, as compared to 105 μg/mg.

### EXAMPLE 24

General Procedure In-vitro Release of the Active from the Microparticles:

Accurately weighed formulations (˜10-25 mg; actual buprenorphine content of ˜20-80 μg of buprenorphine per mg of the microspheres) are suspended in 25 mL of phosphate buffer, pH 7.4, in a volumetric flask and stirred at 37° C.±2° C. with a stirring bar. The entire solution is withdrawn at the desired time intervals (1-7 days) through a syringe filter. The fresh dissolution medium is added through the same filter, and the contents are maintained at 37° C.±2° C. under constant stirring until the next sampling point. The samples are analyzed by HPLC to determine in-vitro buprenorphine HCl and buprenorphine base release from the microspheres.

### EXAMPLE 25

### Expt. 31, MPI #990401R

Scale up of Example 23 with 10% Target Drug Load with Different Lots of PLGAs and Active:

Microcapsules/microspheres were prepared using combination of 50/50 poly (DL-lactide-co-glycolide) of BPI and using solvent evaporation technique as in Example 21 to verify the process variability with different lots of key materials. The 720.0 mg of polyvinyl alcohol (PVA) was weighed and transferred into a 100 mL beaker containing 72 mL of phosphate buffer, pH 7.4 (Solution I). The suspension was stirred until dissolved. Another 3600.0 mg of PVA was weighed in a 17000 mL beaker, and 3600 mL of phosphate buffer (pH 7.4) was added. The suspension was stirred until dissolved. Approximately 80 mL of the solution was kept aside for rinsing the following polymer mixture. PLGA polymers, 1620 mg (Viscosity, 0.66 dL/g and Mw 53,600) and 1620 mg (Viscosity, 0.2 dL/g and Mw 9,440), were mixed and transferred into a 100 mL beaker. Added 12 mL of CH₂Cl₂ to the beaker. The mixture was stirred and sonicated until polymers were dissolved. Buprenorphine free base, which was prepared in Example 22 was weighed accurately (368.5 mg) and added to the beaker containing the PLGA polymer solution. Solution mixing was the same as in example 21. The suspension was kept overnight at 25° C. and stirred at 425 RPM overnight. The microspheres were allowed to settle for one hour. The microspheres were then centrifuged for 15 min. at 3600 rpm using a Mistral centrifuge. The supernatant liquid was decanted, and the microspheres were washed 3 times with nanopure water. The microspheres were transferred into suitable containers, frozen for ˜3 h in the lyophilization chamber and then lyophilized.

The yield of lyophilized microspheres was 3328.7 mg. The microspheres yield was 93%. The percentage incorporation of buprenorphine in the microspheres was analyzed by HPLC and was found to be 85.0 μg of buprenorphine/mg of Microspheres, as compared to ˜100 μg/mg.

Although the present invention has been described in some detail by way of illustration and example for purposes of clarity and understanding, it will be obvious that certain changes and modifications may be practiced within the scope of the claims. Those skilled in the art will be able to ascertain using no more than routine experimentation, many equivalents of the specific embodiments of the invention described herein. These and all other equivalents are intended to be encompassed by the following claims.

### REFERENCES

1. Brewster, D.; Humphrey, M. J.; Mcleavy, M. A. J. Pharm. Pharamcol., 33, 500-6 (1981).
2. Ravis, W. R.; Koster, A. S. In Progress in Pharmacology and Clinical Pharmacology, Eds.; Gustav Fischer Verlag, New York, 7,273-287 (1989).
3. Bullingham, R. E.; McQuay, H. J.; Deyer, D.; Allen, M. C.; Moore, R. A. Br. J. Clin. Pharmacol., 12, 117-22 (1981).
4. Foley, K. M.; Inturrissi, C. E. In Advances in Pain Research and Therapy, Ed.; Raven: New York, 8, 117-127 (1986).
5. Eriksen, J.; Jensen, N. H.; Kamp-Jensen, M.; Bjarno, H.; Friis, P.; Brewster, D. J. Pharm. Pharmacol., 41, 803-805 (1989).
6. Cha, Y.; Pitt, C. G. J. Cont. Rel., 7, 69-78 (1988).
7. Cohen, S.; Yoshioka, T.; Lucarelli, M.; Hwary, L.; Langer, R. Pharm. Res., 8, 713-20 (1991).
8. Schwope, A. D.; Wise, D. L.; Howes, J. F. Life Sci., 17, 1877-86 (1975).
9. Ogawa, Y.; Yamamoto, M.; Okada, H.; Shimamoto, T. Chem. Pharm. Bull., 36, 2576-81 (1988).
10. Ogawa, Y.; Yamamoto, M.; Okada, H.; Yashiki, T.; Shimamoto, T. Chem. Pharm. Bull., 36, 1095-1130 (1988).
11. Mason, N.; Thies, C.; Cicero, T. J. Pharm. Sci., 65, 847-50 (1976).
12. Thies, C.; Bissery M. Biodegradable Microspheres for Parenteral Administration; in Lim F (ed): Biomedical applications of microencapsulation. Boca Raton, Fla., CRC Press, Inc. 1984, pp 53-74.
13. Klement. W.; Kindgen-Milles, D. Pharm. Res., 9, 1233 (1992).
14. Radwan, M. Drug Dev. Ind. Pharm., 20, 2753-62 (1994).
15. Anderson, F. D.; Archer, D. F.; Harman, S. M. Pharm. Res., 10, 369-380 (1993).
16. Johnston, T. P.; Miller, S. C. J. Parent. Sci. Technol., 39, 83-88 (1985).
17. Shintani, S.; Yamazaki, M.; Nakamura, M. Toxicol. Appl. Pharmacol., 11, 293 (1967).
18. Katakam, M., Ravis, W. R., Banga, A. J. J. Cont. Rel. (in press)
19. Ravis, W. R. Statistical Considerations in Percutaneous Absorption Studies, Eds. W. G. Reifenrath and B. W. Kamppainen, Methodology for Skin Absorption, CRC Publications (1990).
20. Proost, J.; H. J. Pharm. Sci., 74, 793-794 (1985).
21. Ravis, W. R.; Ganjam, V. K.; Comerci, C. Biopharm. Drug. Disp., 7, 407 (1986).
22. Cutler, D.; J. J. Pharmacok. Biopharm., 6, 227-241(978).
23. French Patent 456, 824 (1913).
24. Kulkarni, R. K.; Pani, K.; Neuman, C.; Leonard, F. Arch. Surg., 93, 839-43 (1966).
25. Scneider, A. K. French Patent, 1,478,694 (1967).
26. Vissscher, G. E.; Robison, R. L.; Maulding, H. V.; Fong, J. W.; Pearson, J. E.; Argentieri, G. J. J. Biomed. Mater. Res., 19, 349-65 (1985).

1. A pharmaceutical formulation for extended release of buprenorphine from microspheres, said formulation made by steps comprising:
admixing PLGA having a first specific viscosity with PLGA having a second specific viscosity to form a PLGA mixture;
admixing the PLGA mixture with a halogenated organic solvent to form a PLGA-halogenated organic solvent mixture;
admixing the PLGA-halogenated organic solvent mixture with buprenorphine to form a buprenorphine-PLGA-halogenated organic solvent mixture;
admixing a buffered aqueous solution of PVA with the buprenorphine-PLGA-halogenated organic solvent mixture to form an emulsion comprising microspheres, said microspheres comprising buprenorphine;
recovering at least one of said microspheres from the emulsion.

2. A pharmaceutical formulation according to claim 1, wherein the buprenorphine with which the PLGA-halogenated organic solvent mixture is admixed comprises buprenorphine free base.

3. A pharmaceutical formulation according to claim 2, wherein the buprenorphine with which the PLGA-halogenated organic solvent mixture is admixed consists essentially of buprenorphine free base.

4. A pharmaceutical formulation according to claim 1, wherein the buffered aqueous solution of PVA comprises phosphate.

5. A pharmaceutical formulation according to claim 1, wherein the concentration of PVA in the buffered aqueous solution of PVA is about 0.1% (w/v).

6. A pharmaceutical formulation according to claim 1, wherein the pH of the buffered aqueous solution of PVA is between about 6.8 and about 8.0.

7. A pharmaceutical formulation according to claim 6, wherein the pH of the buffered aqueous solution of PVA is about 7.4.

8. A pharmaceutical formulation according to claim 4, wherein the buffered aqueous solution of PVA comprises at least one of the group consisting of sodium phosphate and potassium phosphate.

9. A pharmaceutical formulation according to claim 1, wherein the first specific viscosity is between about 0.01 and about 0.31 dL/g and the second specific viscosity is between about 0.40 and 0.88 dL/g.

10. A pharmaceutical formulation according to claim 9, wherein the first specific viscosity is between about 0.12 and about 0.20 dL/g and the second specific viscosity is between about 0.48 and 0.80 dL/g.

11. A pharmaceutical formulation according to claim 10, wherein the first specific viscosity is between about 0.14 and about 0.18 dL/g and the second specific viscosity is between about 0.56 and 0.72 dL/g.

12. A pharmaceutical formulation according to claim 11, wherein the first specific viscosity is about 0.16 dL/g and the second specific viscosity is about 0.64 dL/g.

13. A pharmaceutical formulation according to claim 1, wherein the halogenated organic solvent comprises dichloromethane.

14. A pharmaceutical formulation according to claim 13, wherein the halogenated organic solvent consists essentially of dichloromethane.

15. A pharmaceutical formulation according to claim 1, wherein the admixing of the buffered aqueous solution of PVA with the buprenorphine-PLGA-halogenated organic solvent mixture comprises sonication.

16. A formulation according to claim 1, wherein the recoverning comprises at least one of the group consisting of sedimentation and lyophilization.

17. A process for making a pharmaceutical formulation for extended release of buprenorphine from microspheres, said process comprising:
admixing PLGA having a first specific viscosity with PLGA having a second specific viscosity to form a PLGA mixture;
admixing the PLGA mixture with a halogenated organic solvent to form a PLGA-halogenated organic solvent mixture;
admixing the PLGA-halogenated organic solvent mixture with buprenorphine to form a buprenorphine-PLGA-halogenated organic solvent mixture;
admixing a buffered aqueous solution of PVA with the buprenorphine-PLGA-halogenated organic solvent mixture to form an emulsion comprising microspheres, said microspheres comprising buprenorphine;
recovering at least one of said microspheres from the emulsion.

18. A process according to claim 17, wherein the buffered aqueous solution of PVA comprises at least one of the group consisting of sodium phosphate and potassium phosphate.

19. A process according to claim 17, wherein the buprenorphine consists essentially of buprenorphine free base.

20. A method of treating a mammal in which treatment with buprenorphine is indicated, said method comprising the step of administering to the mammal a pharmaceutically effective quantity of buprenorphine-containing microspheres prepared by a process comprising:
admixing PLGA having a first specific viscosity with PLGA having a second specific viscosity to form a PLGA mixture;
admixing the PLGA mixture with a halogenated organic solvent to form a PLGA-halogenated organic solvent mixture;
admixing the PLGA-halogenated organic solvent mixture with buprenorphine to form a buprenorphine-PLGA-halogenated organic solvent mixture;
admixing a buffered aqueous solution of PVA with the buprenorphine-PLGA-halogenated organic solvent mixture to form an emulsion comprising microspheres, said microspheres comprising buprenorphine;
recovering at least one of said microspheres from the emulsion.
